(19) United States
(12) Patent Application Publication
(10) Pub. No.: US 2012/0083458 A1
(43) Pub. Date: Apr. 5, 2012

(54) METHODS AND COMPOSITIONS FOR DETECTING AND TREATING INFLAMMATORY DISEASE

(76) Inventors: Detlef Schuppan, Mainz (DE); Yury Popov, Brookline, MA (US); Miroslaw Kornek, Brookline, MA (US)

(21) Appl. No.: 13/237,501

(22) Filed: Sep. 20, 2011

Related U.S. Application Data
(60) Provisional application No. 61/386,232, filed on Sep. 24, 2010.

(51) Int. Cl. A61K 38/17; A61P 1/16; A61P 29/00; G01N 33/566

(52) U.S. Cl. 514/21.92; 435/7.24; 514/1.1

(57) ABSTRACT

The invention features methods of diagnosing inflammatory disease based on the elevated presence microparticles (MP) expressing certain receptors. The invention also features methods of decreasing fibrosis in the liver by administering MP to subjects with liver fibrosis.

### CROSS-REFERENCE TO RELATED APPLICATIONS

This application claims the benefit of the filing date of U.S. Provisional Patent Application No. 61/386,232, filed Sep. 24, 2010.

### STATEMENT AS TO FEDERALLY FUNDED RESEARCH

This work was supported by grant number NIH 1R21DK075857-01A2 from the United States National Institutes of Health. The Government has certain rights in the invention.

### BACKGROUND OF THE INVENTION

Cirrhosis is the consequence of many forms of chronic liver diseases and is characterized by replacement of liver tissue by fibrosis, scar tissue, and regenerative nodules. Liver transplantation, which often remains the only viable treatment option, is only available for a fraction of patients in need, mainly due to the growing demand for transplants in view of an increasing shortage of donor organs. Therefore, there is an urgent need for antifibrotic treatments, which can prevent, halt, or even reverse advanced fibrosis.

In recent years, significant progress has been made in our understanding of fibrosis in general and liver fibrosis in particular. Liver fibrosis can be viewed as a dynamic process, characterized by a preponderance of extracellular matrix (ECM) production, i.e., fibrogenesis, over its degradation, i.e., fibrolysis, which finally leads to distortion of the hepatic architecture (cirrhosis) and loss of organ function.

In hepatic fibrosis, the excessive ECM is produced by activated mesenchymal cells which resemble myofibroblasts. They derive from quiescent hepatic stellate cells (HSC) and periportal or perivenular fibroblasts, here collectively termed HSC. Activation of HSC by several profibrogenic cytokines and growth factors, especially by TGF-β1, is a general feature of fibrosis progression. These factors are mainly produced by activated macrophages or cholangiocytes, but also by liver infiltrating lymphocytes, as shown recently for CD8+ T cells.

Activated HSC can also release pro-inflammatory chemokines/cytokines that attract and activate inflammatory cells, such as MCP-1, IL-6, and TGFβ1. Furthermore, a proinflammatory milieu, e.g., via TNFα and INFγ, can induce adhesion molecules on HSC that further attract inflammatory cells, such as CD54 (ICAM-1) or VCAM-1, the expression of chemokines like CXCL9 and CXCL10, and of chemokine receptors like CXC3R1.

Several studies suggest that even advanced experimental and, possibly, human liver fibrosis can regress once pathogenic triggers are eliminated and sufficient time for recovery is available. Interestingly, the same cells that drive fibrogenesis (HSC) can become major effectors of fibrolysis, e.g., via production and activation of certain matrix metalloproteinases (MMPs). This has been shown in vitro when dermal fibroblasts are plated from a 2D cell culture dish into a 3D collagen gel. Thus under 3D conditions activated fibroblasts/myofibroblasts contract and upregulate MMP production, while procollagen I, the major component of scar tissue is downregulated. However, relevant triggers of myofibroblast or HSC fibrolytic activation remain largely unknown.

One study suggests lymphocytes can modulate fibroblasts in a different, non-cytokine mediated manner. A crude microparticle (MP) preparation released from membranes of Jurkat T cells (an immortal lymphoma T cell line) during activation and early apoptosis could induce synovial fibrolytic MMP expression in fibroblasts. However, it remains unclear how these MP exerted their fibrolytic effects.

### SUMMARY OF THE INVENTION

In one aspect, the invention features a method of diagnosing a subject for an inflammatory disease (e.g., hepatitis, hepatitis C, non-alcoholic steatohepatitis, celiac disease, inflammatory bowel disease, and other inflammatory diseases) by determining the amount of microparticles derived from T cell and other inflammatory cell subsets, including but not limited to CD4+ and/or CD8+ T cells, iNKT cells, CD14+ monocytes/dendritic cells, CD15+ neutrophils, or CD41+ platelets in a blood sample of the subject, where an elevated amount of microparticles derived from these cells diagnoses the subject as having a disease that is dominated or influenced by one or more of T cells, iNKT cells, CD14+ monocytes/dendritic cells, CD15+ neutrophils, or CD41+ platelets.

In the foregoing aspect, the method can further include isolating the microparticles from the blood sample prior to determining the amount of microparticles, e.g., derived from CD4+ and/or CD8+ T cells, iNKT cells, CD14+ monocytes/dendritic cells, CD15+ neutrophils, or CD41+ platelets in the blood.

In any of the foregoing aspects, the determination of the amount of microparticles derived from, e.g., CD4+ and/or CD8+ T cells, iNKT cells, CD14+ monocytes/dendritic cells, CD15+ neutrophils, or CD41+ platelets in the blood sample can include measuring the amount of CD4+ and/or CD8+ cell, iNKT cells, CD14+ monocytes/dendritic cells, CD15+ neutrophils, or CD41+ platelets microparticles in the blood sample or isolated microparticles (e.g., by contacting the blood sample with antibodies to CD4 and/or CD8, Valpha24Vbeta11, CD14, CD15, or CD41).

In another aspect, the invention features a kit for diagnosing an inflammatory disorder including at least one binding agent (e.g., an antibody or

antibody fragment) and instructions for measuring the amount of microparticles derived from particular cell types in a blood sample of the subject, where the amount of microparticles derived from particular cell types diagnoses the subject as having an inflammatory disease associated with said particular cell type. The at least one binding agent including a binding agent specific for one or more of the following cell types: CD4+ and/or CD8+ T cells, CD14+ monocyte or dendritic cells, invariant chain natural killer (iNKT) T cells, and/or CD41+ platelet cells

In another aspect, the invention features a pharmacological composition including insolated microparticles (e.g., synthetic microparticles, microparticles isolated from a human, or microparticles isolated from a T cell line like Jurkat cells), wherein the microparticles include receptors or membrane bound molecules found on CD4 and/or CD8 T cells and in addition or in the alternative include CD54 and/or CD147 receptors (e.g., recombinant receptors).

The foregoing pharmaceutical compositions can further include siRNA against at least one gene selected from the group consisting of procollagens I, III, IV, V, VI, HSP47, TGF beta1, TGFbeta2, PDGF-B, CTGF, TGF beta receptors I, II and III, PDGFbeta receptor, integrins alpha1beta1, alpha2beta1, alpha3beta1, alpha5betal, MCP-1, CXCL4, CCL2, and CXCR2.

In another aspect, the invention features a method of treating liver fibrosis in a subject by administering any of the foregoing pharmaceutical compositions.

Included is also the isolation and expansion of autologous or heterologous (other donor than the patient) T cells from peripheral blood, e.g., via CD3 (CD8) affinity chromatography, negative selection or other standard T cell isolation procedures, followed by PHA, cytokine driven or other standard nonspecific or specific in vitro T cell expansion methods, with the aim of generating and purifying of large numbers of homogeneous MP in vitro that will be then infused into the patient as therapy. This method will take advantage of autologous tissue histocompatibility to reduce any potential side-effects and will allow repeated treatments.

By “blood sample” is meant a blood, serum, or plasma specimen obtained from a patient or a test subject.

By “treating” is meant administering a pharmaceutical composition for prophylactic and/or therapeutic purposes or administering treatment to a subject already suffering from a disease (e.g., fibrosis of the liver) to improve the subject's condition or to a subject who is at risk of developing a disease. In the case of liver fibrosis, treatment would result in an increase (e.g., by at least 5%, 10%, 25%, 50%, 75%, 100%, 200%, 500%, or more) in fibrolysis or a reduction (e.g., by at least 5%, 10%, 25%, 50%, 75%, or more) of overall fibrosis or fibrogenesis, or fibrosis or fibrogenesis in a particular region of the organ.

By “elevated” is meant an amount of MP in a sample that is at least 5%, 10%, 25%, 50%, 75%, 100%, 200%, 500%, or more, greater than that measured in a control sample (e.g., from a healthy subject).

By “decreased” is meant an amount of MP in a sample that is at least 5%, 10%, 25%, 50%, 75%, or less, than that measured in a control sample (e.g., from a healthy subject).

By “inflammatory disease” is meant a disease characterized by specific T cell, dendritic cell/monocyte (CD14+), neutrophil (CD15+) or platelet (CD41+)) responses.

### BRIEF DESCRIPTION OF THE DRAWINGS

FIG. 1 is a diagram showing T cell-derived membrane associated molecules (e,g., signaling receptors), including EMMPRIN (CD147) are transferred to HSC membranes via shedded MR These MP fuse with the HSC membrane which is facilitated by CD54. The transferred receptors can activate novel signaling pathways or auto-/paracrine signaling loops in HSC that favor a switch towards a fibrolytic phenotype via, e.g., MAP kinase and/or NFkB pathway activation and subsequent induction of MMPs and other proteases mediating fibrolysis or fibrogenesis inhibition.

FIG. 2A is a pair of graphs showing the amount of Annexin V staining and CD3-APC staining in individual cells. These graphs are representative of FACS analysis of CD3-APC and Annexin V-FITC double positive S100-MP in a human plasma sample from a healthy donor.

FIG. 2B is a pair of graphs showing the relative percentage of circulating CD3 and Annexin V double positive S100-MP from patients with hepatitis C and normal ALT (<40 IU/L; n=4) as compared to patients with chronic hepatitis C and elevated ALT (>40 IU/L; n=14). Patients with hepatitis C and normal ALT (<40 IU/ml) have significantly lower numbers of T cell MP than patients with hepatitis C and high ALT levels (>100 IU/ml, n=8) (*p<0.05.

FIG. 2C is a pair of graphs showing percentage of CD4/Annexin V and CD8/Annexin double positive S100-MP are significantly higher in the plasma of patients with ALT>100 11IU/L (n>9) compared to healthy controls and HCV patients with ALT<40 IU/L (n>9; *p<0.05, **p<0.005, respectively).

FIG. 2D is a graph showing CD8+ S100-MP are ˜80% positive for CD25, a common T cell activation marker.

FIG. 3A is a graph showing FACS analysis demonstrating that S100-MP are Annexin V FITC high and CD3 APC high, whereas the S10-MP fraction is Annexin V FITC low and CD3 APC low.

FIG. 3B is a pair of graphs showing mean fluorescence intensity (MFI) for the indicated marker, which is 11-fold higher for Annexin V and 8-fold higher for CD3 on S100-MP compared to S10-MP; analysis of n=4 events; means±SD; *p<0.0001 and **p=0.004. MH values from S100-MP and S10-MP obtained from apoptotic (ST), PHA-activated and apoptotic (ST & PHA), or PHA-activated Jurkat T-cells (PHA).

FIG. 3C is a photomicrograph showing ultrastructural analysis of the two subtractions of MP generated from apoptotic Jurkat T cells. MP were fractionated as described below and subjected to electron microscopy. The S100-MP fraction is composed of vesicles surrounded by a double layered plasma membrane, whereas S10-MP are more heterogeneous containing numerous electron dense cell debris; magnification×51,000.

FIG. 3D is a graph showing sidescatter profiles of events in blood plasma samples after isolation of S100-MP and with addition of 3 μm beads and intact T cells for standardization.

FIG. 4A is a series of graphs showing FACS analysis demonstrating CD3 receptor transfer from S 100-MP to HCS. 200,000 LX-2 HSC were incubated with 100,000 Jurkat T cell-derived S 100-MP. CD3 (APC) positive LX-2 HSC were quantified after 6 hours. Unstained HSC and HSC incubated with 0.04 μm/mL ST served as controls.

FIG. 4B is a graph showing time dependent uptake of CD3 S 100-MP by HSC as assessed by FACS analysis, demonstrating maximal MP-uptake (15-17%) after 6 hours; from n=3 events; means±SD; *p=0.003 and **p=0.01.

FIG. 4C is a series of photomicrographs showing fluorescence microscopy confirming S100-MP uptake and membrane fusion with HSC. S100-MP were labeled with PKH26 membrane dye and incubated with LX-2 HSC. After 30 min MP had attached to HSC membranes in a punctate pattern. At 60 min the red-fluorescent signal increased while being more diffusely distributed over the surface of the HSC indicating more extensive MP fusion with HSC membranes.

FIG. 5A is a series of photomicrographs showing S10-MP labeled with PKH26 membrane dye and added to HSC. S10-MP remained a particulate fraction that was only loosely associated with the HSC, in contrast to S100-MP which merged with HSC membranes (see FIG. 4C).

FIG. 5B is a series of graphs showing Annexin V and against 7-AAD staining using FACS analysis demonstrating a lack of significant apoptosis induction in HSC 24 hours after incubation with S100-MR In contrast, ST, at a dose that reflects maximal possible ST contamination in MP preparations, induced apoptosis.

FIG. 5C is a graph showing quantitative analysis of the 7-AAD/Annexin FACS data for late stage apoptosis, showing a 7-fold higher percentage of late apoptotic HSC after exposure to ST (0.04 μM/mL) compared to S100-MP (*p=0.047).

FIG. 6 is a series of graphs showing mRNA transcript levels of the indicated gene incubated with the indicated medium: “medium” refers to plain medium;

“ST” refers to 0.04 μM/mL staurosporine, and “MP” refers to S10-MP or S-100-MP from apoptotic Jurkat T cells suspended in 350 μL medium for 24 hours.

FIG. 7 is a graph showing MMP-3 transcript levels were determined by quantitative RT-PCR in primary rat HSC (200,000 cells per well in 12-well plates) that were incubated with S10-MP or S100-MP (2,000× or 50,000 MP per well) generated from apoptotic Jurkat I cells for 24 hours. Medium only served as control. Results (means±SD) are expressed as arbitrary units relative to beta2-microglobulin mRNA. *p<0.03 vs. medium control.

FIG. 8 is a series of graphs showing mRNA transcript levels of the indicated MMP as determined by quantitative RT-PCR in LX-2 cells (200,000 cells each well in 12-well plates) that were incubated with S10-MP or S100-MP (1,000 or 50,000) from activated and apoptotic Jurkat T cells for 24 hours. ST (0.04 μM/mL) or plain medium served as controls. All experiments were at least performed twice with n=3-4 per group. Results (means±SD) are expressed as arbitrary units relative to beta2-microglobulin mRNA; *p<0.05 vs. medium control.

FIG. 9 is a series of graphs showing mRNA transcript levels of the indicated gene in TGFβ1 (5 ng/mL)-activated HSC when incubated with the indicated amount of S100-MP for 24 hours. All experiments were performed at least twice with n=3-4 per group. Results (means±SD) are expressed as arbitrary units relative to beta2-microglobulin mRNA; *p<0.05 vs. medium control.

FIG. 10 is a series of graphs showing mRNA transcript levels of the indicated MMP as determined by quantitative RT-PCR in LX-2 cells (200,000 cells in mL in 12-well plates) that were incubated with 1,000 or 50,000 S10-MP or S100-MP from PHA-activated human CD4+ T cells for 24 hours. PHA (0.05 μg/mL) or plain medium served as controls. Experiments were performed twice with n=3 per group. Results (means±SD) are expressed as arbitrary units relative to beta2-microglobulin mRNA; *p<0.05 vs. medium control.

FIG. 11 is a series of graphs showing mRNA transcript levels of the indicated MMP as determined by quantitative RT-PCR in LX-2 cells (200,000 cells each well in 12-well plates) that were incubated with S10-MP or S100-MP (1,000 or 50,000) from apoptotic CD8+ T cells for 24 hours. ST (0.04 μM/mL) or plain medium served as controls. All experiments were at least performed 2-3 times with n=3-4 per group. Results (means±SD) are expressed as arbitrary units relative to beta2-microglobulin mRNA; *p<0.05 vs. medium control.

FIG. 12 is a series of graphs showing mRNA transcript levels in LX-2 HSC (200,000 cells/ml per well) of the indicated gene in cells incubated with S10-MP or S100-MP (1,000 or 50,000) from PHA-activated and apoptotic CD8+ T cells for 24 hours, Measurements were collected using quantitative RT-PCR. ST (0.04 μM/mL) or plain medium (medium) served as controls. Results (means±SD) are expressed as arbitrary units relative to beta2-microglobulin mRNA; *p<0.05 vs. medium control.

FIG. 13A is a series of graphs showing CD11a and Annexin V staining using FACS analysis of S100-MP. Approximately 64% of MP were double stained.

FIG. 13B is a series of graphs showing CD54 staining using FACS analysis in HSC cells stimulated with TNFα (10 ng/mL) for 0, 4, and 24 hours resulting in a 40% upregulation of CD54 (*p<0.001).

FIG. 13C is a graph showing mRNA transcript levels of the indicated MMP gene in HSC after addition of S100-MP with or without TNFα (10 ng/mL) for 24 h (*p<0.05, **p=0.04, ***p=0.001).

FIG. 13D is a graph showing mRNA transcript levels of the indicated MMP gene in HSC after incubation with a CD54 blocking antibody (50 μg/mL) or an IgG-matched control antibody for 2 hours, followed by addition of S100-MP for 24 hours. MMP-3 and -13 transcripts were determined by quantitative PCR. CD54-blocking significantly decreased MMP-3 and MMP-13 induction by 40-45% (*p=0.02 and **p=0.046). All experiments were at least performed twice or more with n=3 per group. Results (means±SD) are expressed as arbitrary units relative to beta2-microglobulin mRNA.

FIG. 14A is a series of graphs showing CD147 and CD3APC staining in S100-MP and LX-2 HSC by FACS analysis. CD147 is a candidate membrane molecule on T cell MP to trigger MMP expression in HSC.

FIG. 14B is a graph showing mRNA transcript levels of the indicated MMP gene in an experiment where CD8+ T cell-derived S100-MP (PHA+ST treatment) are incubated with CD147 blocking antibody (50 μg/mL) for 1 hour, followed by addition to LX-2 HSC for 24 hours. CD147 blocking significantly decreased. MMP-3 and MMP-9 induction in HSC as determined by quantitative PCR (by 35%, *p=0.007 and 30%, **p=0.03, respectively). Experiments were performed twice with n=3 per group. Results (means±SD) are expressed as arbitrary units relative to beta2-microglobulin mRNA.

FIG. 14C is a graph showing unchanged mRNA transcript levels of MMP-3 in HSC treated with the P13 kinase inhibitor LY294002 (LY, 5 μg/mL). Complete abrogation of MMP-3 induction by the ERKI1/2 inhibitor U0126 (U, 5 μgi/mL), and 50% inhibition by the p38 kinase inhibitor SB203580 (SB, 5 μg/mL ) and the proteasome (NFκB) inhibitor MC132 (MG, 15 μg/mL) (*p=0.02), as compared to untreated S100-MP stimulated controls.

FIG. 14D is a photo micrographs showing nuclear translocation of NF-κB p65 in LX-2 HSC exposed to S100-MP from Jurkat T cells for 60 minutes. Representative out of three similar experiments is shown.

FIG. 15 is a series of graphs showing the percentage of S100-MP from cells positive for the indicated marker that were isolated from the plasma of patients with the indicated diseases and healthy controls. (*p<0.05, **p<0.005, vs. healthy controls).

FIG. 16 is a series of graphs showing the percentage of S100-MP from cells positive for the indicated marker that were isolated from the plasma of celiac patients with the indicated diseases stage as indicated compared to healthy controls. Unique MP profiles were obtained for celiac patients with active celiac disease compared to celiac disease with remission or with mild activity. Here, percentages of CD8 T cell derived S100-MP were elevated in patients with active vs. mild celiac disease or celiac disease in remission (*p<0.05, **p<0.005, vs. healthy controls), serving as an interesting serum/plasma marker of celiac disease activity which has not been available to date.

FIG. 17 is a series of graphs showing the percentage of S100-MP cells positive for the indicated marker in 1 mL plasma as compared to serum. Serum and plasma were obtained from four normal control subjects at the same time, frozen and thawed once, and subjected to MP analysis. Yield of CD4+ and CD8+ MP showed no significant differences between serum and plasma, indicating that retrospective studies from stored serum (plasma) samples can be performed.

### DETAILED DESCRIPTION OF THE INVENTION

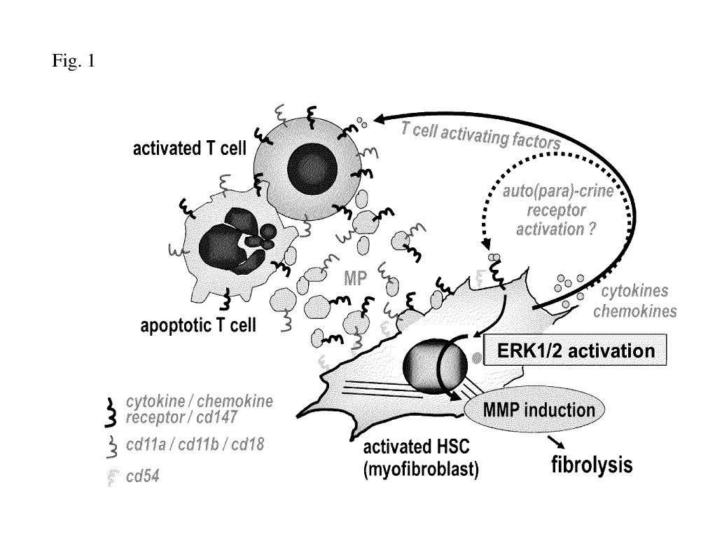

In general, the invention features methods of diagnosing the overall inflammatory activity and profile of inflammatory diseases (e.g., hepatitis C or NASH, as well as of other diseases that are characterized by a specific T cell, or dendritic cell/monocyte (CD14+), neutrophil (CD15+) or platelet (CD41+)) response, based on the relative presence of the respective microparticles (MP). The invention also features methods of decreasing fibrosis in the liver by administering CD8+ (CD4+) MP to subjects with liver fibrosis. The invention is based on the discovery that blood samples taken from subjects with, e.g., hepatitis C, NASH, celiac disease, IBD (and other diseases characterized by significant inflammation and T cell turnover) contain characteristically elevated (or decreased) levels of CD4+, CD8+, iNKT, CD14, or CD15 MP. Further, administration of CD8+ MP is effective to induce fibrolysis in in vitro models of liver fibrosis. The proposed mechanisms are schematically illustrated in FIG. 1.

### Diagnostic Methods

The invention features methods of diagnosing diseases based on the presence and features of MP in subject samples (e.g., blood samples). MP bear the cell surface receptors of the blood cells (e.g., T cells) from which they derive. Therefore, the presence of MP with certain surface markers is indicative of the disease and importantly of cell specific disease activity with which the corresponding blood cell (e.g., T cell) is associated.

For example, MPs with CD4 and CD8 markers are diagnostic of hepatitis (e.g., hepatitis C). NASH is also associated with a striking increase in CD14+ (monocyte/dendritic cell) MP. Celiac disease is associated with characteristic changes in CD4+, CD8+ T cell, iNKT and CD41+ (platelet derived) MP. Inflammatory bowel disease is associated with characteristic changes in CD4+ T cell and CD14+ MP.

Diagnosis is based on the relative frequency of MPs in a subject sample (e.g., a human, mouse, rat, dog, or cat sample) associated with the indicated diseases and the severity and prognosis of the disease can be further ascertained by comparing the MP levels with control levels (e.g., as taken from a healthy subject, or a sample from a subject that, retrospectively, is deemed to have a severe or mild form of the indicated disease).

Diagnosis can be based on the detection of unique MP profiles for patients with chronic hepatitis C (HCV), non-alcoholic steatohepatitis (NASH), various activities of celiac disease, and inflammatory bowel disease (IBD). For example, percentages of CD8 T cell derived S100-MP were elevated in active HCV infection (ALT>100 IU/mL) and NASH, but unchanged in mild HCV infection (ALT<40 IU/ml), in celiac disease and to a lesser degree in successfully treated IBD (the latter two being CD4 T cell dominated diseases as compared to viral hepatitis which is both CD4 and CD8 T cell dominated). Percentages of CD4 T cell derived S100-MP were significantly increased in active HCV infection, NASH and celiac disease. CD41 (platelet-derived) MP were decreased in NASH, celiac disease and IBD, whereas CD15 (neutrophil)-derived S100-MP were non significantly decreased in NASH and celiac disease, but significantly reduced in IBD patients. CD14 (monocyte/dendritic cell-derived) MP were strongly reduced in active HCV infection, mildly increased in IBD and highly increased in NASH. Percentages of invariant chain natural killer (iNKT) T cell (Valpha24/Vbeta11 double positive) derived MP were significantly increased in NASH, celiac and IBD patients. Thus each investigated disease is characterized by an individual pattern of cell specific MP, which can be analyzed by FACS and used as an early diagnostic tool to assess the cellular pattern and intensity of the respective immune activation in the blood.

Measurement of transmembrane proteins (e.g., CD4 or CD8) in MP can be performed directly on a subject sample or upon MP particles isolated from a subject sample. Transmembrane proteins, carbohydrate/glycosaminoglycan/proteoglycan, or lipid/glycolipid/lipoprotein structures can be detected by, for example, contacting the sample or isolated MP with a transmembrane specific antibody (e.g., a fluorescently, peroxidase, streptavidin or luminescent labeled antibody, or a HLA (MHC)-tetramer/pentamer). Antibody (tetramer/pentamer) bound to MP can be detected and quantified, e.g., using FACS analysis, via overall fluorescence or luminescence measurement, or microscopically. MP can also be sorted according to the labeled transmembrane protein, carbohydrate/glycosaminoglycan/proteoglycan, or lipid/glycolipid/lipoprotein structure and subject to quantitative analysis for specific proteins, carbohydrates/glycosaminoglycans/proteoglycans, lipids/glycolipid/lipoproteins, or RNAs and DNAs in the MP.

Methods of isolation MP from subject samples are described herein (e.g., differential centrifugation, antibody or aptamer affinity chromatography with positive or negative selection).

### Methods of Treatment

The invention features methods of treating liver disease by administering CD4+ or preferably CD8+ MP (e.g., CD4+ CD8+ MP). These MP can be generated from cells derived from the subject to be treated (e.g., autologous cells) or from other cells and cell lines. Large quantities of MP can be generated ex vivo from T cells by use of agents that activate T cells (e.g., phytohemagglutinin) or induce apoptosis (e.g., UV light, staurosporin, fas ligand, or fas activating antibody).

The treated T cells (e.g., Jurkat cells) can be engineered or further treated to express the desired markers and active principles (e.g., CD54, CD147, antifibrotic/fibrolytic proteins, carbohydrates/glycosaminoglycans/proteoglycans, lipids/glycolipid/lipoproteins, or RNAs and DNAs). Such expression can be obtained through, e.g., the introduction of recombinant constructs. In one embodiment, the Jurkat cells are not activated prior to induction of MP formation.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment.

Routes of administration for the various embodiments include, but are not limited to, topical, transdermal, nasal, and systemic administration (such as, intravenous, intramuscular, subcutaneous, inhalation, rectal, buccal, vaginal, intraperitoneal, intraarticular, ophthalmic, otic, or oral administration).

Indications for the therapy of the invention includes any liver disease associated with liver fibrosis including cirrhosis of the liver due to alcohol consumption, hepatitis associated with viral infection, nonalcoholic steatohepatitis, autoimmunity, haemochromatosis, congenital, immune mediated or acquired disease, Wilson's disease, cystic fibrosis and other genetic or congenital biliary and nonbiliary liver diseases, post transplant fibrosis, Budd-Chiari syndrome, and hepatocellular carcinoma.

### Experimental Results

T Cell Derived Microparticles Circulate in the Blood Plasma of Healthy Controls and are Increased in Patients with Active Hepatitis C

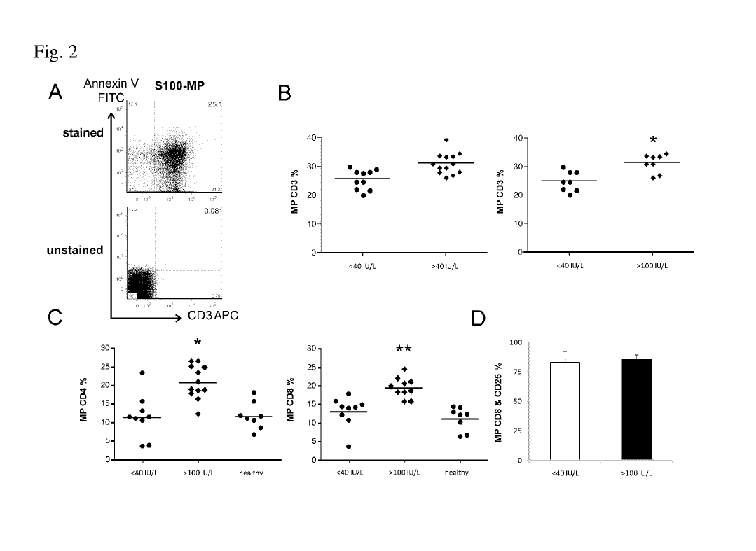

We searched for T cell derived microparticles (MP) in human plasma from normal controls and patients with chronic hepatitis. Using a two-step centrifugation at 10,000 and 100,000 g, we focused on S100-MP. FACS analysis using the MP marker Annexin V and the general T cell marker CD3 showed that indeed T cell derived MP were present in human blood plasma (FIG. 2A) and that their numbers in blood plasma increased significantly from 25% in healthy controls and patients with serologically mild hepatitis C (ALT<40 IU/mL) to 31% in patients with serologically active hepatitis C (ALT>100 IU/mL) (FIG. 2B). The higher numbers of T cell MP were paralleled by a higher mean fluorescence intensity (MFI) for the CD3 marker (FIG. 2C). Furthermore, looking at T cell subsets, patients with active hepatitis C had a significant increase in circulating MP derived from CD4+ as well as CD8+ T cells (1.8- and 1.4-fold, respectively) (FIG. 2D). Finally, 80% of CD8+ MP were additionally CD25+, an accepted T cell activation marker.

### Isolation and Characterization of T Cell Derived Microparticles

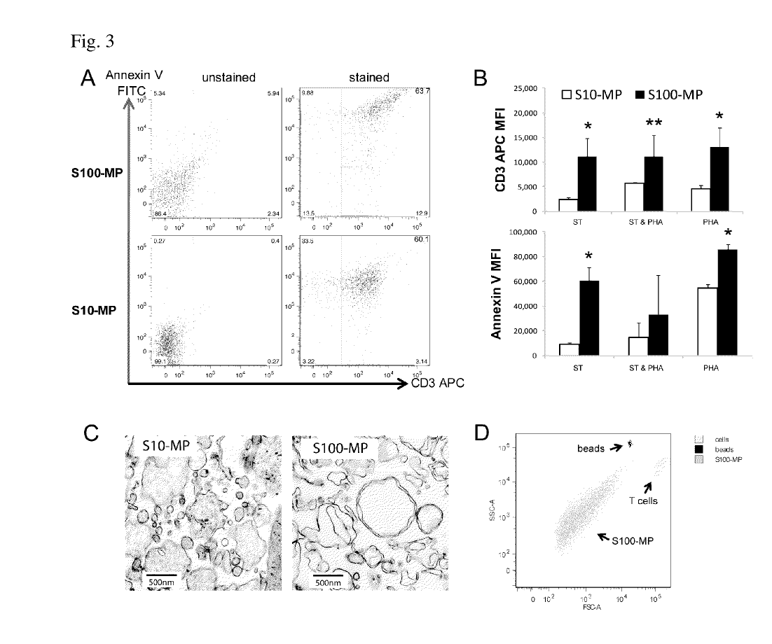

Due to the low numbers of circulating MP, initial characterization and functional analyses were performed with T cell MP generated from the human Jurkat T cell line (that expresses CD4) and from peripheral blood T cells of healthy human donors. We stimulated MP release either by activation using phytohemagglutinin (PHA), or by induction of apoptosis using the tyrosine kinase inhibitor staurosporine (ST). The S10-MP fraction was Annexin V-low and CD3-low, and the S100-MP fraction was Annexin V-high and CD3-high (FIG. 3A), which was confirmed by analysis of MFI (FIG. 3B). This difference between S100-MP and S10-MP was found irrespective of the mode of generation of MP (by PHA, by ST, or by PHA and ST combined, FIG. 3B). Electron microscopic images from both fractions demonstrated that S10-MP were heterogeneous in size and contained electron dense material, indicating debris of intracellular organelles, while S100-MP mostly showed a more homogeneous structure, being surrounded by a double layered cell membrane and were electron-lucent, with a variable diameter ranging from 30-700 nm (FIG. 3C). FIG. 3D shows a typical FACS scatter plot that characterizes the S-100 MP along with 3 μm marker beads and intact T cells which were added for standardization. In the following, we focused on the characterization of S100-MP, using S10-MP as negative controls.

CD3 T Cell Receptor Transfer from S100-MP to Cell Membranes of Human Hepatic Stellate Cells

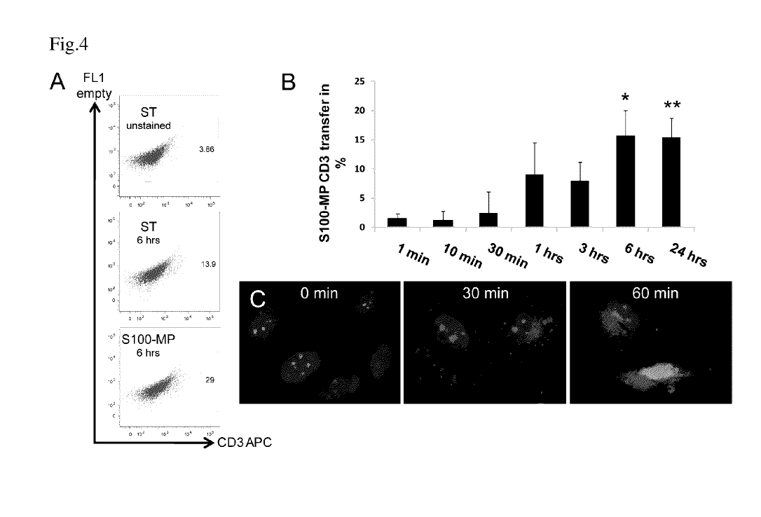

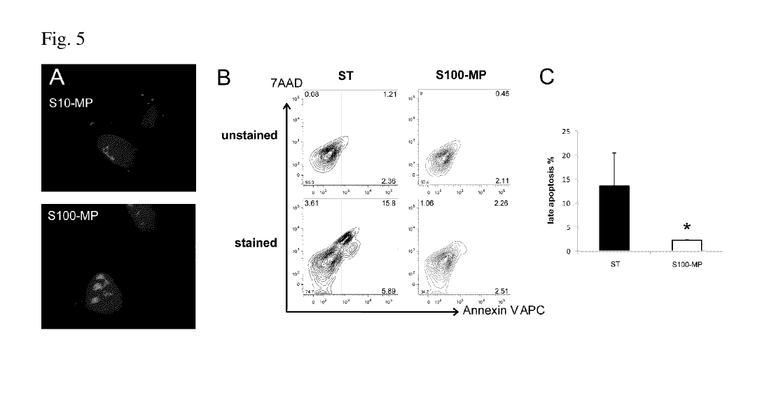

The exclusive expression of transmembrane CD3 on T cells allowed us to monitor the transfer of CD3 (and likely other transmembrane molecules) from S100-MP to human LX-2 hepatic stellate cells (HSC). FACS analysis demonstrated that after six hours of incubation with S100-MP, the transfer of CD3 from MP to HSC peaked, with 17% of the HSC being positive for CD3 (FIG. 4A). FIG. 4B shows the time-dependent increase of CD3 transfer from S100-MP to HSC, with minimal CD3 transfer at 30 min and 8-9% and 15-17% CD3 positive HSC between 1-3 hrs and 6-24 hrs, respectively. In support of the FACS data, fluorescence microscopy demonstrated that S100-MP labeled with the membrane-dye PKH26 began to attach to HSC membranes at 30 min, generating a punctate red-fluorescent membrane pattern. At 60 min and beyond a diffuse membrane staining, indicative of membrane fusion was observed (FIG. 4C). Membrane fusion was not found with PKH26-labelled S10-MP (FIG. 5A).

### Effect of S100 T Cell-MP on Fibrosis-Related Gene Expression by HSC

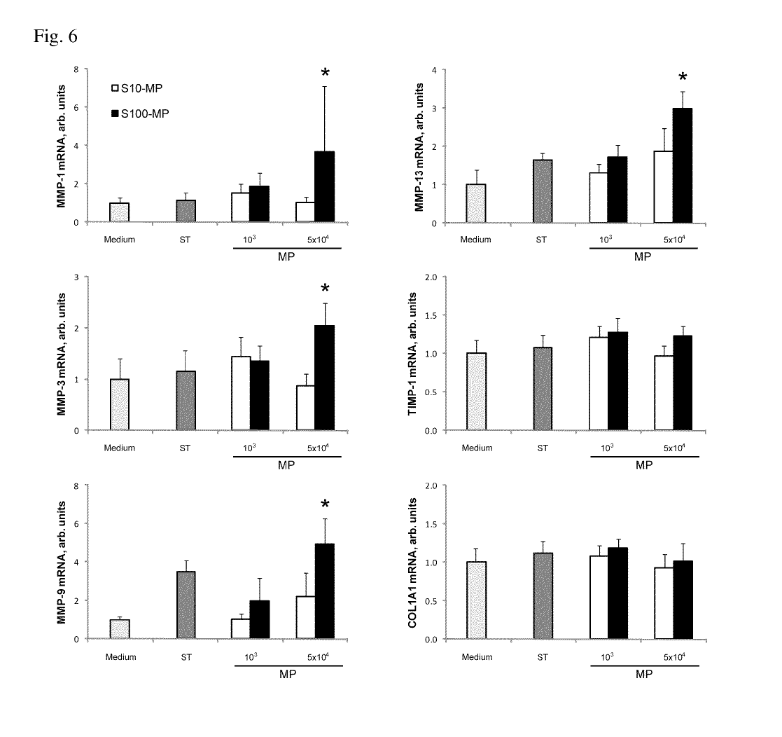

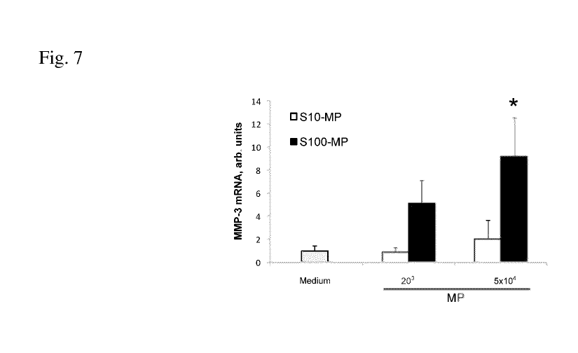

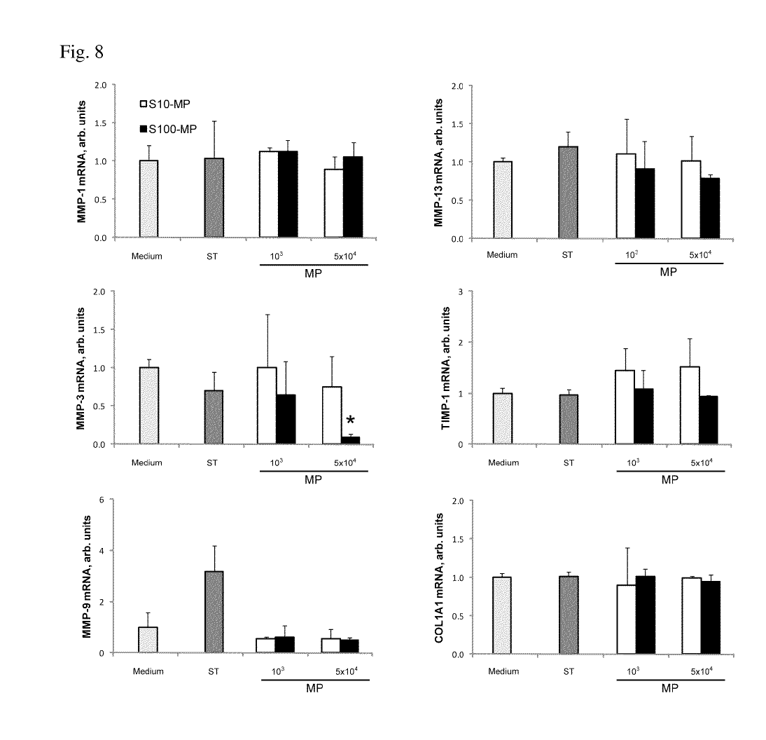

Fibrosis related transcripts were measured from 200×10³ serum-starved human LX-2 HSC 24 hours after addition of 1×10³ or 50×10³ S100-MP from Jurkat T cells using quantitative RT-PCR. S10-MP, plain medium, and ST alone served as controls. MP were obtained from PHA-activated and/or apoptotic (ST-treated) Jurkat T cells. After T cell apoptosis induction, significant changes in fibrosis-related transcripts were found with 50×10³ S100-MP, while equivalent amounts of S10-MP had no effect (FIG. 6). Twenty four hours after addition, S100-MP induced a significant (2.05-4.9-fold) upregulation of fibrolytic genes (MMP-1, -3, -9, -13) in HSC, whereas ST alone induced only MMP-9, and transcript levels of the profibrogenic genes TIMP-1 and procollagen α1(I) were unaffected (FIG. 6). Similar results were obtained when S100-MP were incubated with freshly isolated primary rat HSC. Here the human S100-MP induced MMP-3 even 9-fold (FIG. 7). S100-MP from apoptotic T cells that had been preactivated by PHA did not induce upregulation of MMPs in human HSC, but rather downregulated MMP-3 (FIG. 8). A similar response was found with S100-MP that were derived from merely PHA-activated T cells, indicating that only Jurkat T cells that underwent apoptosis without prior activation generated putatively fibrolytic MP. Equivalent amounts of S100- or S10-MP from Huh-7 hepatoma cells made apoptotic with ST were lacking any fibrolytic induction potential on HSC.

### T Cell Derived S100-MP do not Induce Apoptosis of HSC

It was reported earlier that MP derived from macrophages could trigger apoptosis in recipient cells. Since it is known that matrix metalloproteinases (MMPs), especially MMP-3 is upregulated in cells undergoing apoptosis and since our data show that indeed S100-MP derived from apoptotic T cells prominently upregulated MMP-3 in HSC, we evaluated apoptosis induction by S100-MP using Annexin V externalization and 7-amino-actinomycin D (AAD) labeling as readout. Jurkat T cell-derived 5100-MP did not induce enhanced apoptosis or necrosis in HSC after 24 hours of incubation, whereas HSC that were treated with ST alone exhibited up to 14% necrosis and 10% late apoptosis (FIGS. 5B and 5C), which, in addition, ruled out significant ST contamination in our MP preparations.

### S100-MP Abrogate HSC Profibrogenic Responses to TGFβ1

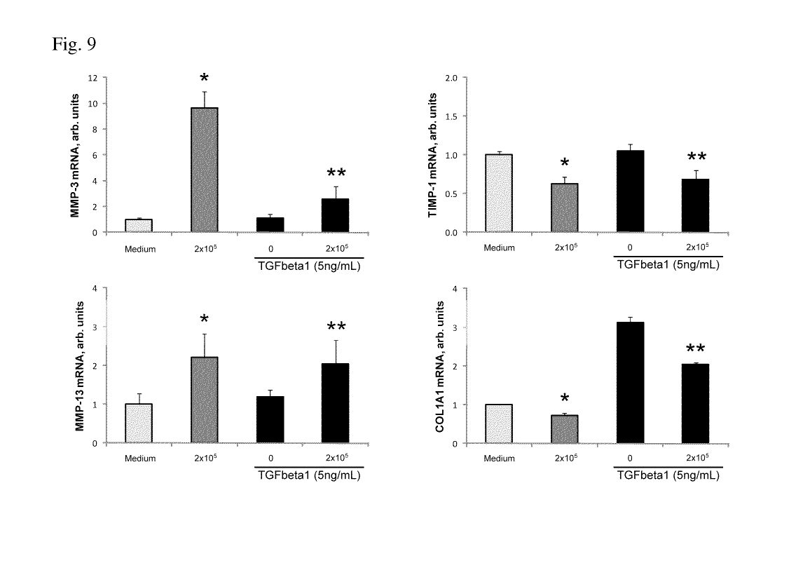

Human HSC were exposed to 5 ng/mL TGFβ1, which elicits a strong fibrogenic response. Jurkat T cell-derived S100-MP did not only blunt the TGFβ1 response, by reducing procollagen α1(I) expression, but even induced fibrolytic MMP transcripts beyond the levels produced by unstimulated HSC (FIG. 9). Thus TGFβ1 enhanced HSC procollagen α1(I) expression 2.7-fold, which after MP addition was reduced by almost 40%, and MP increased the expression of MMP-3 and MMP-13 almost 2.5- and 2.1-fold, respectively. In addition, both in TGFβ1-treated and -untreated HSC, the addition of S100-MP significantly reduced profibrogenic TIMP-1 expression by 30-35% (FIG. 9).

Comparison of the Effect of S100-MP Derived from CD4+ and CD8+ T Cells

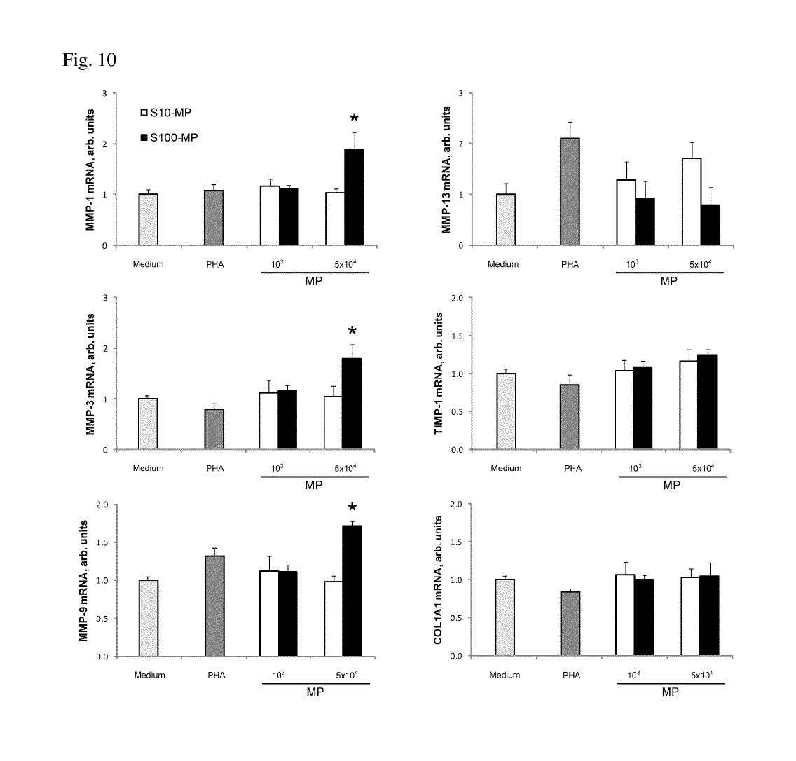

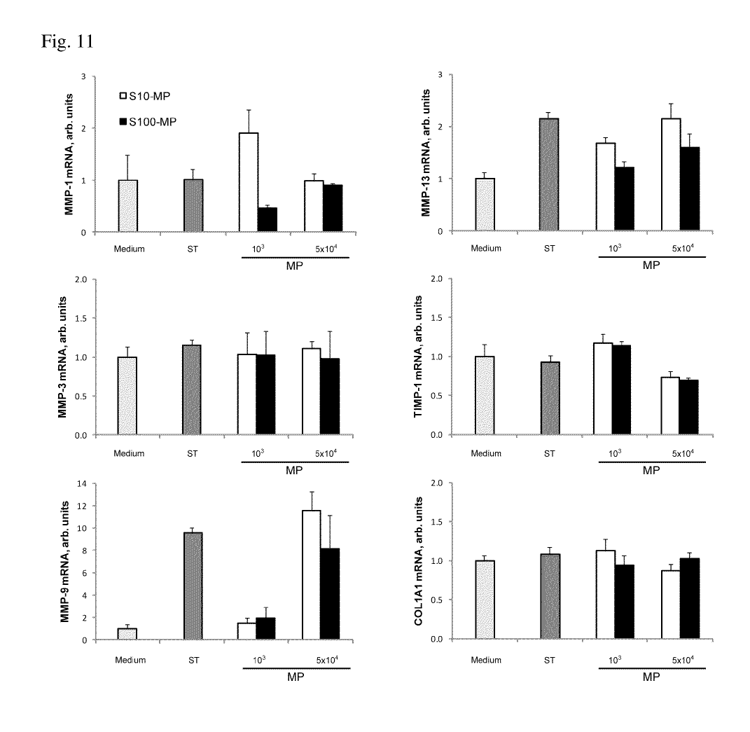

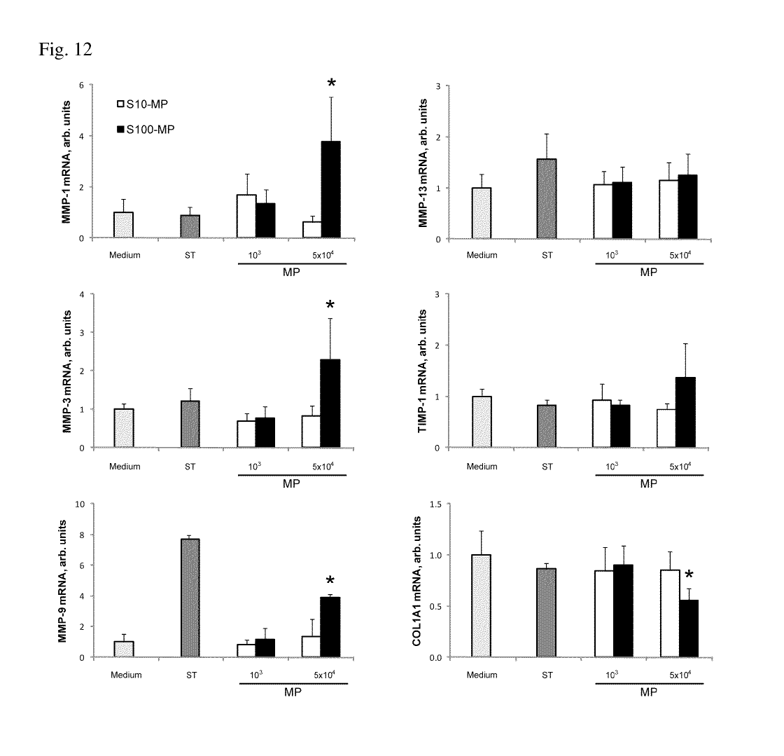

S100-MP were produced and purified from peripheral T cells of healthy donors. Overall, apoptotic CD4+ T cell-derived MP induced MMP expression in HSC much less efficiently than MP from CD8+ T cells, irrespective of their mode of generation (with or without prior activation by PHA). Thus MP from CD4+ T cells did not significantly affect MMP-1, -3, -9, -13, TIMP-1 or procollagen α1(I) expression. If MP shedding was induced only by CD4+ T cell activation with PHA, a significant induction was observed for MMP-1, MMP-3, and MMP-9 mRNA (between 1.7- and 3-fold), while procollagen α1(I) and TIMP-1 transcript levels remained unchanged (FIG. 10). S100-MP derived from apoptotic CD8+ T cells did not affect fibrosis related gene expression (FIG. 11). However, S100-MP from apoptotic CD8+ T cells that were pre-activated by PHA produced the strongest fibrolytic effects in HSC (FIG. 12). Their addition increased HSC MMP-1, MMP-3, and MMP-9 mRNA 3.8-, 2.3-, and 3.9-fold, respectively, while MMP-13 and TIMP-1 transcript levels remained unaffected. Of note, procollagen α1(I) mRNA was reduced significantly by 45%. In line with these findings, S100-MP derived from CD8+ T cells that were only pre-activated by PHA (without subsequent apoptosis induction), increased MMP-1 transcripts 1.9-fold and reduced procollagen α1(I) transcripts 30%. Taken together and as summarized in Table 1, fibrolytic effects were mainly induced by MP from activated CD8+>CD4+ T cells, in contrast to MP from the apoptotic Jurkat T cell line.

TABLE 1
Summary of observed fibrolytic effects on human hepatic stellate cells induced
by S100-MP derived from activated and/or apoptotic human T cells.

| | Jurkat | Jurkat | Jurkat | CD4+ | CD4+ | CD4+ | CD8+ | CD8+ | CD8+ |
|---|---|---|---|---|---|---|---|---|---|
| | (ST) | (PHA & ST) | (PHA) | (ST) | (PHA & ST) | (PHA) | (ST) | (PHA & ST) | (PHA) |
| MMP-1 | (++) | ~ | ~ | ~ | ~ | + | ~ | ++ | ++ |
| MMP-3 | ++ | ~~~ | ~~ | + | ~ | + | ~ | ++ | ~ |
| MMP-9 | ++ | ~ | ~ | ~ | ~ | + | (+++) | +++ | (++) |
| MMP-13 | ++ | ~ | ~ | ~ | ~ | ++ | + | ~ | ~ |
| TIMP-1 | ~ | ~ | ~ | ~ | ~ | ~ | ~ | ~ | ~ |
| Procollagen | ~ | ~ | ~ | ~ | ~ | ~ | ~ | ~~ | ~ |

MMP-1, -3, -9, -13, TIMP-1, and procollagen α1(I) transcript levels were determined by quantitative RT-PCR in LX-2 HSC (200,000 cells each well) incubated with (active) S-100 or (inactive) S-10 MP for 24 hours. T cells were activated with PHA at day 1 and day 8. Apoptosis was induced by ST at day 9. MP were isolated as described before. Only effects >50% were considered relevant and upregulation categorized as follows: +++, >4-fold, ++, >2-fold; <2-fold compared to the medium control; ( ): not significant towards the PHA + ST control.

### CD54 (ICAM-1) Dependent Uptake of S100-MP

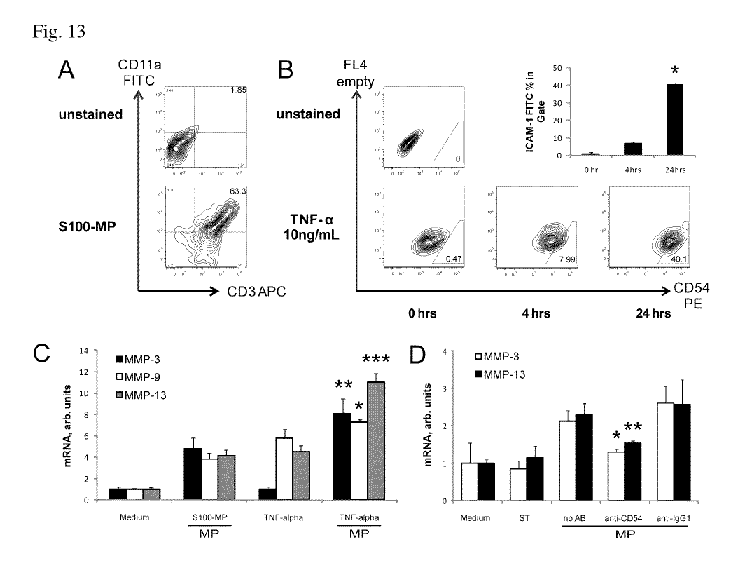

It remained to be shown what cell membrane molecule(s) or receptor(s) mediate(s) attachment and uptake of S100-MP by HSC. CD54 is expressed by HSC and upregulated by proinflammatory signals. Our FACS analysis revealed that >60% of S100-MP were highly positive for the CD54 ligand CD11a (FIG. 13A). Assuming that ICAM-1 on the recipient HSC is engaged by CD11a/CD18 on the S100-MP, any treatment of HSC that increases CD54 should enhance MP uptake and subsequent fibrolytic activation of HSC. We therefore incubated HSC with 10 ng/mL TNF-α, a strong inducer of CD54, which induced a robust (>10-fold) upregulation of CD54 after 24 hrs (FIG. 13B). This led to a further significant MP-induced increase (by 40%) of MMP-3 mRNA expression in the induced HSC as compared to untreated HSC (FIG. 13C). A direct effect of TNF-α on HSC could be ruled out, since TNF-α alone was not capable to enhance HSC MMP-3 mRNA, and alone modestly induced HSC MMP-9 and MMP-13 expression. For MMP-3 the effect of combined TNF-α and MP treatment was overadditive as compared to the added effects of TNF-α or S100-MP alone (FIG. 13C).

To corroborate that the observed effects were indeed due to an engagement of CD54 on HSC, HSC were incubated with CD54-blocking antibody or an isotype matched control antibody 2 hours prior to addition of S100-MP. CD54-blocking resulted in a significant downregulation of MMP-3 and MMP-13 induction by MP from Jurkat T cells (40% and 45%, respectively) as compared to HSC pre-incubated with the control antibody (FIG. 13D), confirming the engagement of CD54 in MP uptake by recipient HSC.

### Emmprin (CD147) is Involved in MP-Induced MMP Induction in HSC

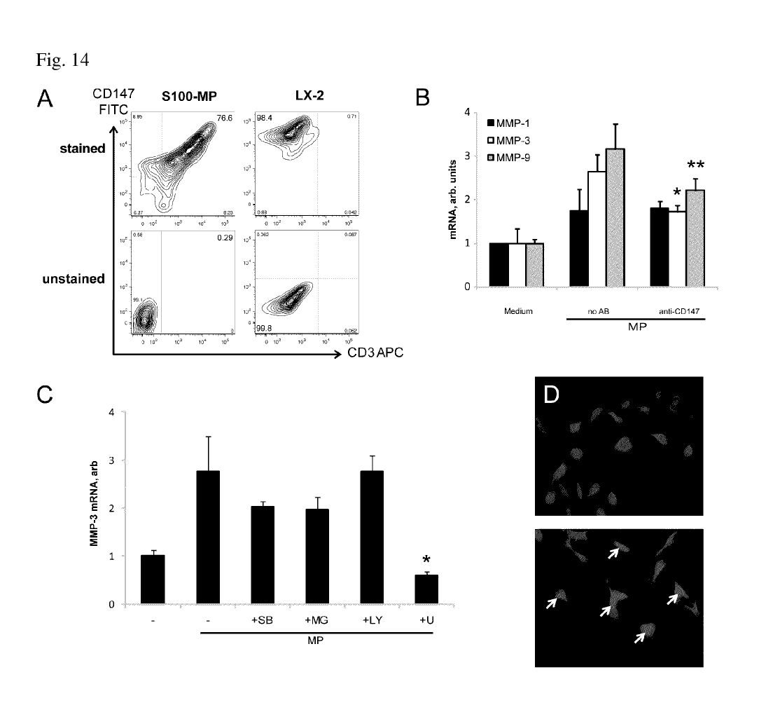

In order to identify (cell membrane) molecules in MP that could be implicated in the fibrolytic activation of HSC, either as ligands or as (transmembrane) signal transducing receptors, we performed proteomic analysis of S100-MP from apoptotic Jurkat T cells, with S100-MP from apoptotic Huh-7 hepatoma cells serving as negative controls. Comparative quantitative proteomics using iTRAQ isobaric tagging yielded three candidate cell-associated molecules, other than growth factor or cytokine receptors, namely Nomo-1 and Nomo-2 (molecules involved in the inhibition of TGFβ signaling, and Emmprin/Basigin (CD147) (Table 2). CD147 has been described as an inducer of MMPs, mainly MMP-1, MMP-2, MMP-3, MMP-9 and MMP-11. Of note, CD147 is activated by encounter of two CD147 positive cells, leading to homodimerization via cell-cell binding. Accordingly, FACS analysis showed that Jurkat-derived S100-MP as well as HSC were highly positive for CD147 (>70% and 99%, respectively) (FIG. 14A). Blocking of CD147 by pre-incubating S100-MP (CD8+ T cell derived after induction with PHA and ST) with anti-CD147 resulted in a significant reduction of MMP-3 and MMP-9 mRNA (35% and 30%, respectively) compared to addition of S100-MP alone (FIG. 14B), indicating that CD 147 contributes significantly to fibrolytic activation of HSC, but that additional molecules may be involved.

TABLE 2
Selection of proteins identified in purified
T cell derived MP by proteome analysis

| | Intracellular/ | Cell membrane |
|---|---|---|
| Nuclear | cytoskeletal | associated |
| PR domain Zn-finger | Alpha/beta/gamma | CD45 |
| protein 5 | actin | |
| NFAT-1 | Rho-A/C/G | 34/67 kD Laminin |
| | | receptor |
| Leucin rich repeat | Ezrin/Radixin/Moesin | Na/K ATPase |
| protein 6 | | |
| Storkhead box protein | HSP70/75/90 | HLA-IA*3 |
| 1 | | |
| Transcr. elong. factor- | Cytokeratin-9 | GTPalpha S |
| 5 | | |
| Histone-1/-2/-4 | Ras GTPase activating | GTPgamma2 |
| | protein | |
| Elongation factor- | Nima related protein | CD147/Emmprin/Basigin |
| 1alpha | kinase | |
| Y-box transcription | Rab7b | Nomo-1 & -2 |
| factor BP | | |
| | Cofilin-1 | Annexin-6/Lipocortin-6 |
| | MEK-11 | Clathrin heavy chain |
| | Tubulin-alpha/beta3 | Glycophorin C |
| | Ubiquitin | Thyroid hormone rec. |
| | | assoc. protein |

S100-MP proteins were extracted from apoptotic Jurkat T cells and Huh-7 hepatoma cells as negative controls, digested with trypsin and labeled with isobaric tags. Tagged tryptic digests were pooled, peptides fractionated by ion exchange and HPLC analysis, and differential protein expression analyzed by MALDI-TOF mass spectroscopy as described. Shown is a selection of most abundant proteins specifically expressed on S100-MP from T cells.

### Fibrolytic Activation of HSC by S100-MP Depends of NF-κB & ERK1/2 Pathways

In order to define major signaling pathways that lead to MMP-induction by S100-MP, we used specific inhibitors of several kinases. MP-stimulated MMP-3 mRNA expression served as fibrolytic read-out. MMP-3 expression was completely abrogated by inhibition of p42/p44 MAP kinase (ERK1/2), while inhibition of phosphatidyl-inositol-3 (PI3) kinase/Akt did not affect MMP-3 transcript levels, and inhibition of p38 and NF-κB signaling resulted only in a modest MMP-3 mRNA suppression by 28% (FIG. 14C). >10% of HSC showed NF-κB relocation to the nucleus after incubation with S100-MP, confirming minor activation of the NF-κB pathway (FIG. 14D).

Association of MP with Other Diseases

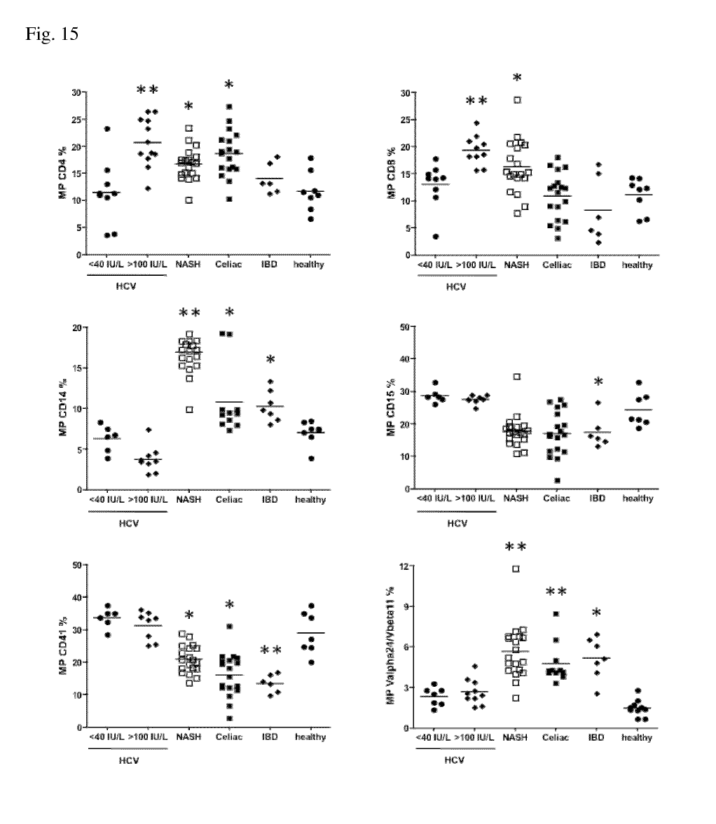

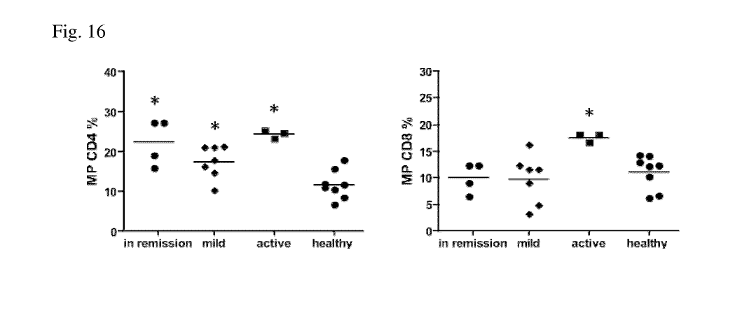

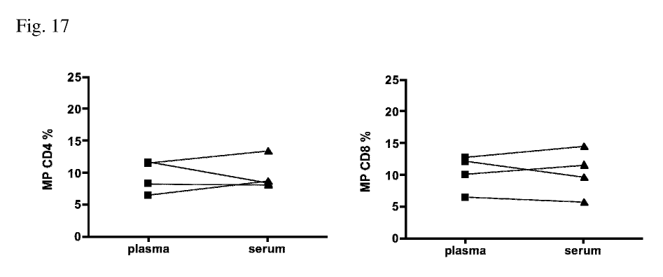

FIG. 15 shows data of samples obtained patients with HCV, celiac disease, NASH and inflammatory bowel disease (IBD). Each sample was tested for levels of S-100 MP derived from CD4+ and CD8+ T cells, CD14+, CD15+, CD41+ and iNKT cells. FIG. 16 demonstrates that for celiac disease, MP levels are highest in patients with active disease. FIG. 17 shows that plasma and serum samples can both be used to reliably determine the levels of CD8+ and CD4+ MP.

### Methods

### Cell Lines

Human Jurkat T cells (ATTC #: CRL-2570) were from ATCC (Manassas, Va.). Cells were grown in 10% fetal calf serum (FCS) in RPMI medium (with 5% CO2 in a humidified atmosphere. LX-2 human HSC were grown in 2.5% FCS in DMEM. Cells were split every 3 days at a 1:3 ratio. All media were from Cellgrow® (Manassas, Va.).

### Lymphocyte Isolation

Human peripheral blood was collected in heparinized tubes from healthy volunteers within a protocol approved by the Children's Hospital, Boston, that provides anonymized blood samples. Mononuclear cells were isolated by centrifugation over Ficoll-Paque™ Premium (GE Healthcare, Uppsala, Sweden). After three washes in HBSS cells were resuspended in 10% FCS in RPMI. CD4+ and CD8+ T cells were isolated using negative selection magnetic cell sorting beads (Miltenyi Biotec, Auburn, Calif.).

Isolation of T Cell Microparticles from Plasma of Patients with Hepatitis C and Healthy Controls

Human peripheral blood was collected in citrate containing tubes from anonymized patients and healthy controls within a protocol approved by the Beth Israel Deaconess Medical Center, Boston. MP were isolated according the established protocol by differential centrifugation and the number of S100-MP was characterized by FACS w/t and with staining for Annexin V in conjunction with CD3, CD4, CD8 and CD25 (eBioscience™, San Diego, Calif.) as detailed below.

Quantification of Microparticles from Plasma of Patients with Hepatitis C, NASH, Celiac Disease, Inflammatory Bowel Disease and Healthy Controls

Human peripheral blood was collected in citrate containing tubes from anonymized patients and healthy controls within protocols approved by the Beth Israel Deaconess Medical Center, Boston. MP were isolated and standardized as to their number as above, and the relative percentage of cell specific MP determined by FACS using antibodies to CD4, CD8, CD14, CD15, CD41 (all from eBioscience™, San Diego, Calif.) and the invariant chain Valpha24/Vbeta11 (BioLegend™, San Diego, Calif. and BD Biosciences Pharmingen™, San Diego, Calif.).

Stimulation of MP Release from T Cells by Inducing Apoptosis and/or Activation

For induction of apoptosis T cells were cultured in RPMI and treated with 4 μM/mL Staurosporine (ST, Cell Signaling Technology®, Danvers, Mass.) for 4 hours. T cells were activated with 5 μg/mL Phytohemagglutinin-M (PHA, Roche, Mannheim, Germany) for 24 hours, and restimulated with PHA after 3 days. During stimulation cultures were supplemented with 5 ng/mL IL-2 (PEPROTECH®, Rocky Hill, N.J.). Three days after restimulation cells were separated from media containing MP by centrifugation at 500 g for 15 min. The cell-free supernatants were then centrifuged at 10×10³ g for 20 min yielding S10-MP, while the resultant supernatant was then centrifuged at 100×10³ g for 90 min to yield purified, biologically active S100-MP.

### Characterization and Quantification of MP Using Flow Cytometry

The MP preparations were characterized on a LSR2 FACS analyzer with CELLQuest software (Becton Dickinson, San Jose, Calif.). Cytometric data was further analyzed with FlowJo 7.2 (Tree Star, Inc., Ashland, Oreg.). Defined populations of particles were gated by forward and sideward scattering (FSC and SSC) acquired from runs including 500 standard beads (Becton Dickinson, San Jose, Calif.) and followed by gating for anti-CD3-APC and AnnexinV-FITC (both eBioscience™, San Diego, Calif.) double positive events. Annexin V staining of MP has previously been validated as a marker for MP. The number of double positive MP was calculated relative to the number of total beads added to the samples. The expression of CD11a and CD147 on MP was assessed using anti-CD11a- and anti-CD147-FITC (eBioscience™, San Diego, Calif.; GeneTex® Inc., Irvine, Calif., respectively).

### Labeling of MP and Tracking Experiments

MP membranes were labeled with the PKH26 lipid dye (Sigma-Aldrich, St. Louis, Mo.) following the manufacturer's instructions. Membrane-labeled S10- and S100-MP were coincubated with LX-2 cells for 0-1, 30 and 60 min, washed extensively and fixed with 2% paraformaldehyde for 15 min at RT. Nuclei were counterstained with the Hoechst 33342 DNA dye (Sigma-Aldrich).

### Quantification of CD3 Receptor Transfer Towards HSC by Flow Cytometry

HSC (200×10³/well) were seeded into six-well cell culture plates (BD Labware, Franklin Lakes, N.J.) for 12 hours, serum-starved for 24 hrs, followed by incubation with 100×10³ S100-MP for 1 min up to 24 hours. After incubation the HSC were washed with PBS, removed from the dishes by a short incubation with trypsin/EDTA for 5 min (0.25% Trypsin, 2.2 mM EDTA in HBSS, Cellgrow®, Manassas, Va.), and washed with FACS buffer. Single cell suspensions were stained with anti-CD3-APC in FACS buffer and CD3 receptor transfer was quantified using FACS analysis as described above.

Incubation of HSC with T Cell-Derived MP and Quantitative PCR

HSC (200×103/well) were seeded into six-well cell culture plates and serum-starved as above. HSC were then incubated with 1×10³ or 50×10³ S10-MP or S100-MP for 24 hours, followed by total RNA extraction from cells using TRIzol (Invitrogen, Carlsbad, Calif.). One μg of RNA was reverse-transcribed using random primers and Superscript RNase H-reverse transcriptase (Invitrogen). The sequences of primers and probes for transcripts related to fibrogenesis or fibrolysis are listed in Table 3. Target genes were mainly transcripts encoding MMPs that are capable of degrading fibrous tissue (MMP-1, 3, 9, 13) vs. COL1A1 (procollagen α1(I)) and the prominent MMP-inhibitor TIMP-1. Relative transcript levels were quantified by real-time RT-PCR on a LightCycler 1.5 instrument (Roche, Mannheim, Germany) using the TaqMan methodology as described previously (52). TaqMan probes (dual-labeled with 5′-FAM and 3′-TAMRA) and primers were designed using the Primer Express software (Perkin Elmer, Wellesley, USA), synthesized at Eurofins MWG Operon (Huntsville, Ala., USA), and validated as described by us. Experiments were performed in triplicates and values represent means±SD, being expressed as arbitrary units relative to the housekeeping gene beta2-microglobulin.

TABLE 3
Primers and probes used for quantitative RT-PCR

| gene | sense | anti-sense | probe |
|---|---|---|---|
| hMMP-1 | CAG TGG TGA TGT TCA GCT AGC | GCC GAT GGG CTG GAC A | CAT CCA AGC CAT ATA TGG ACG TTC CCA |
| | TCA | | AA |
| hMMP-3 | GTT CCG CCT GTC TCA AGA TGA | GGG ACA GGT TCC GTG GGT A | TAA ATG GCA TTC AGT CCC TCT ATG GAC |
| | | | CTC C |
| hMMP-9 | ACT CGC GTG TAC AGC CGG | AGG GAT ACC CGT CTC CGT G | CCG CGA CAC CAA ACT GGA TGA CG |
| hMMP-13 | TGG CAT TGC TGA CAT CAT GA | GCC AGA GGG CCC ATC AA | AAG TCG CCA TGC TCC TTA ATT CCA AAA |
| | | | GAG |
| hTIMP-1 | TGT TGT TGC TGT GGC TGA TAG C | TCT GGT GTC CCC ACG AAC TT | TTC TGC AAT TCC GAC CTC GTC ATC AGG |
| hCOL1A1 | CAG CCG CTT CAC CTA CAG C | TCA ATC ACT GTC TTG CCC CA | TCG ATG GCT GCA CGA GTC ACA CC |
| hβ2MG | TGA CTT TGT CAC ACC CCA AGA TA | AAT CCA AAT GCG GCA GCT TC | TGA TGC TGC TTA CAT GTC TCG ATC CCA |
| rMMP-3 | CCG TTT CCA TCT CTC TCA AGA | CAG AGA GTT AGA TTT GGT GGG | AGA TGG TAT TCA ATC CCT CTA TGA ACC |
| | TGA | TAC CA | TCC |
| rβ2MG | CCG ATG TAT ATG CTT GCA GAG | CAG ATG ATT CAG AGC TCC ATA | AAC CGT CAC CTG GGA CCG AGA CAT GTA |
| | TTA A | GA | |

### ICAM-1 Upregulation on HSC by TNFα

TNFα (PEPROTECH®, Rocky Hill, N.J., USA) was added to HSC cultures, and ICAM-1 expression assessed after 2, 4 and 24 hrs by flow cytometric analysis using anti-ICAM-1-FITC (eBioscience™, San Diego, Calif., USA) on a LSR2 FACS analyzer as described above.

### Comparative Proteomic Analysis of S100-MP

S100-MP proteins were extracted from ST-treated Jurkat T cells and Huh-7 hepatoma cells as described above. 20 μg of membrane protein were digested with trypsin and labeled with isobaric tags (4-plex iTRAQ, Applied Biosystems, Foster City, Calif.) following the manufacturer's instructions as described, subjected to two dimensional peptide fractionation and analyzed for the comparative proteomic signature by Matrix-Assisted Laser Desorption Ionization—Time of Flight/Time of Flight Mass Spectrometry.

### CD54 (ICAM-1) and CD147 (EMMPRIN) Blocking Studies

Subconfluent, serum-starved HSC were pre-incubated with monoclonal anti-human CD54 blocking antibody or isotype matched (IgG1) control antibody (GeneTex® Inc., Irvine, Calif., USA) at a final concentration of 50 μg/mL for 120 min, washed and incubated with Jurkat T cell derived S100-MP. S100-MP were incubated with monoclonal anti-human CD147 blocking antibody (Abcam, Cambridge, Mass., USA) or with IgG1 control antibody (GeneTex® Inc., Irvine, Calif., USA) at a final concentration of 50 μg/mL for 60 min, before being added to HSC. The effect on fibrosis-related gene expression in HSC was assessed by quantitative real-time PCR as described above.

### P65 NFκB Translocation

HSC serum-starved for 24 hours were washed with ice-cold phosphate buffer, and fixed in cold methanol for 10 min. Nuclear translocation of p65 NFκB was detected by incubating cells with polyclonal p65 antibody (1:100; Delta Biolabs) for 30 min followed by TRITC-conjugated antirabbit IgG (1:200, Dako, Germany). Representative images were documented using a scanning confocal microscope (Carl Zeiss, Germany).

### Signaling Pathway Inhibition

Pathway inhibition experiments were performed in 24 hour serum-starved HSC. The inhibitors SB203580 (p38 MAPK), U0126 (ERK1/2), and LY294002 (PI3K) (all from LC Labs, Woburn, Mass., USA) were all used at concentrations that efficiently and specifically block the respective kinase pathways in activated HSC as established previously. The proteasome inhibitor MG132 (Rockland Inc., USA) was used to block NFκB nuclear translocation and activity.

### Statistical Analysis

All data are given as arithmetic means with SD. Differences between values of independent experimental groups were analyzed for statistical significance by the two-tailed Student's t-test. An error level (p) <0.05 was considered significant.

### Apoptosis Assay

24 hrs serum-starved HSC were incubated with S100-MP for 24 hrs. Apoptosis and necrosis induction by S100-MP were assessed by FACS analysis for Annexin V and 7-aminoactinomycin D staining (both from eBioscience™, San Diego, Calif., USA) on a LSR2 FACS analyzer with CELLQuest software (Becton Dickinson, San Jose, Calif.).

### Isolation of Primary rat HSC

Primary HSCs were isolated from male Wistar rats (Retired Breeders, 450-500 g, Charles River Laboratories Int., MA, USA) according to a previously published procedure. Animal experimentation was approved by the Institutional Review Board of the Beth Israel-Deaconess Medical Center, Boston. Animals were housed with 12-hour light-dark cycles and with water and standard rat/mouse pellet chow ad libitum. Briefly, the liver was perfused with 0.1% Pronase E and 0.025% type IV collagenase in Dulbecco's modified Eagle's medium for 10-15 min, followed by digestion with 0.04% Pronase, 0.025% collagenase, and 0.002% DNase at 37° C. for 10-30 min and by a two-step centrifugation through a 11% and 13% gradient of Nycodenz at 1,500 g for 15 min. Cell viability was assessed by Trypan Blue exclusion and was routinely greater than 95-98%. Purity of HSC isolates was confirmed by their stellate shape, and cytoplasmic lipid-droplets showing greenish autofluorescence at 390 nm excitation.

Contamination with Kupffer cells, as assessed by the ability to engulf 3-μm latex beads, was 3-5% after isolation and undetectable after 10 days in first passage. Cells were used at 10 days of primary culture. Culture-activated, myofibroblast-like HSC were used between passages 3-5.

### Proteomic Analysis of S100-MP

Twenty μg of membrane protein from ST-treated Jurkat T cells and Huh-7 hepatoma cells were denatured with 0.1% (v:v) SDS and then reduced by addition of 4 mM tris-(2-carboxyethyl)phosphine for one hour at 56° C. Disulfide bonds were blocked by incubation with a final concentration of 8 mM methyl methanethiosulfonate at room temperature for 10 min, followed by digestion with 10 μg Trypsin (Promega, 1 mg/ml) overnight at 37° C. Digests were labeled with the 4-plex iTRAQ isobaric tags, according to the manufacturer's protocol. Before pooling, success of labeling was confirmed by evaluating five of the highest intensity peaks on a mass spectrometer. Tagged tryptic digests were pooled and subjected to two-dimensional peptide fractionation before mass spectrometry to maximize the number of identified peptides. Pooled samples were concentrated by vacuum centrifugation and solubilized in 1 mL 10 mM KH2PO4, 25% Acetonitrile, pH 2.8, for strong cation exchange chromatography over a 4.6×100 mm POROS HS/20 column (Applied Biosystems, Foster City, Calif.) on an 1100/1200 HPLC system (Agilent Technologies, Santa Clara, Calif.) using a two-step KCl gradient at a flow rate of 0.5 mL/min over 50 minutes. Fifteen fractions were selected dried by vacuum centrifugation and resuspended in 100 μl reverse phase buffer A (2% acetonitrile, 0.1% trifluroacetic acid) and each fraction underwent reverse phase chromatography on a Dionex Ultimate NanoLC equipped with an Acclaim C18 PepMap 100 μ-precolumn followed by an analytical nanoflow C18 PepMap 100 column. Peptides were eluted with a 5%-50% gradient of acetonitrile over 60 minutes. All fractions containing peptides, based on UV absorbance at 214 nm, were directly spotted onto AB 4700 OptiTOF MALDI (Matrix-Assisted Laser Desorption Ionization) target plates using a Probot printing robot (Dionex, Sunnyvale, Calif.). Alpha-Cyano-4-hydroxycinnamic acid (CHCA) ionization matrix (Sigma-Aldrich, Saint Louis, Mo.) was mixed with the sample at a 1:2 ratio using an in-line mixing Tee in the Probot. A total of 485 fractions were collected and analyzed on the ABI 4700 MALDI-TOF/TOF MS (Matrix-Assisted Laser Desorption Ionization—Time of Flight/Time of Flight Mass Spectrometer) by tandem mass spectrometry. The 15 most abundant precursors of each spot were fragmented by MS-MS with collision-induced dissociation using medium gas pressure with ambient air. Relative abundance quantitation and peptide and protein identification were performed using GPS Explorer (Applied Biosystems, Software Revision 50861). The Swiss-Prot Homo sapiens protein database was used for all searches. The confidence value for each peptide was calculated based on agreement between the experimental and theoretical fragmentation patterns. Each protein was provided with a confidence score based on confidence scores of its constituent peptides with unique spectral patterns. Each peptide was associated with the quantitative score for each of the iTRAQ tags to calculate the relative expression levels.

### Other Embodiments

Various modifications and variations of the described methods and compositions of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method of diagnosing a subject for an inflammatory disease comprising determining the amount of microparticles derived from particular cell types in a blood sample of said subject; wherein the amount of microparticles derived from particular cell types diagnoses said subject as having an inflammatory disease associated with said particular cell type.

2. The method of claim 1, wherein said inflammatory disease is hepatitis and said microparticles derived from particular cell types are derived from CD4+ and/or CD8+ T cells.

3. The method of claim 2, wherein said hepatitis is hepatitis C.

4. The method of claim 2, wherein said method comprises determining the amount of microparticles derived from CD4+ T cells.

5. The method of claim 2, wherein said determination comprises measuring the amount of CD4+ microparticles in said blood sample.

6. The method of claim 2, wherein said method comprises determining the amount of microparticles derived from CD8+ T cells.

7. The method of claim 2, wherein said determination comprises measuring the amount of CD8+ microparticles in said blood sample.

8. The method of claim 2, wherein said determining the amount of microparticles comprises contacting said blood sample with antibodies to CD4 and/or CD8.

9. The method of claim 1, wherein said inflammatory disease is non-alcoholic steatohepatitis (NASH) and said microparticles derived from particular cell types are derived from CD4+, CD8+, CD14+ monocyte or dendritic cells, invariant chain natural killer (iNKT) T cells, and/or CD41+ platelet cells.

10. The method of claim 1, wherein said inflammatory disease is liver disease, and said microparticles derived from a particular cell type are derived from CD4+, CD8+ or CD14+ monocyte or dendritic cells.

11. The method of claim 1, wherein said inflammatory disease is celiac disease and said microparticles derived from particular cell types are derived from CD4+ and/or CD8+ T cells, CD14+ monocyte or dendritic cells, invariant chain natural killer (iNKT) T cells, and/or CD41+ platelet cells.

12. The method of claim 1, wherein said inflammatory disease is inflammatory bowel disease and said microparticles derived from particular cell types are CD4+ and/or CD8+ T cells, CD14+ monocyte or dendritic cells, invariant chain natural killer (iNKT) T cells, and/or CD41+ platelet cells.

13. The method of claim 1, further comprising isolating or separating the microparticles from said blood sample prior to determining the amount of microparticles derived from a particular cell type.

14. A pharmacological composition comprising isolated microparticles, wherein said microparticles comprise CD4 and/or CD8 receptors.

15. The pharmacological composition of claim 14, wherein said isolated microparticles comprise CD4 and CD8 receptors.

16. The pharmacological composition of claim 14, wherein said isolated microparticles comprise CD8 receptors.

17. The pharmaceutical composition of claim 14, wherein said microparticles further comprise CD54 and/or CD 147 receptors.

18. A pharmaceutical composition comprising isolated microparticles comprising CD54 and/or CD147 receptors.

19. The pharmaceutical composition of claim 14, wherein said isolated microparticles further comprise siRNA against at least one gene selected from the group consisting of procollagens I, III, IV, V, VI, HSP47, TGF beta1, TGFbeta2, PDGF-B, CTGF, TGF beta receptors I, II and III, PDGFbeta receptor, integrins alpha1beta1, alpha2beta1, alpha3beta1, alpha5beta1, MCP-1, CXCL4, CCL2, and CXCR2.

20. The pharmaceutical composition of claim 14, wherein said microparticle receptors are recombinant.

21. The pharmaceutical composition of claim 14, wherein said microparticles are synthetic.

22. The pharmaceutical composition of composition of claim 14, wherein said microparticles are isolated from a human cell, a human cell line, or an animal cell line.

23. The pharmaceutical composition of claim 22, wherein said microparticles are isolated from a human and comprises recombinant CD4 and/or CD8 receptor.

24. A method of treating liver fibrosis in a subject, said method comprising administering to said subject the composition of claim 14.

25. A kit for diagnosing an inflammatory disorder in a subject comprising at least one binding agent and instructions for measuring the amount of microparticles derived from particular cell types in a blood sample of said subject; wherein the amount of microparticles derived from particular cell types diagnoses said subject as having an inflammatory disease associated with said particular cell type; and wherein said at least one binding agent comprises a binding agent specific for one or more of the following cell types: CD4+ and/or CD8+ T cells, CD14+ monocyte or dendritic cells, invariant chain natural killer (iNKT) T cells, and/or CD41+ platelet cells.

26. The kit of claim 26, wherein said at least one binding agent is an antibody or antibody fragment.


SEQUENCE LISTING (27 sequences)

SEQ ID NO: 1 - DNA, 24 bp, Artificial Sequence
  Synthetic Construct
cagtggtgatgttcagctagctca

SEQ ID NO: 2 - DNA, 16 bp, Artificial Sequence
  Synthetic Construct
gccgatgggctggaca

SEQ ID NO: 3 - DNA, 29 bp, Artificial Sequence
  Synthetic Construct
catccaagccatatatggacgttcccaaa

SEQ ID NO: 4 - DNA, 21 bp, Artificial Sequence
  Synthetic Construct
gttccgcctgtctcaagatga

SEQ ID NO: 5 - DNA, 19 bp, Artificial Sequence
  Synthetic Construct
gggacaggttccgtgggta

SEQ ID NO: 6 - DNA, 31 bp, Artificial Sequence
  Synthetic Construct
taaatggcattcagtccctctatggacctcc

SEQ ID NO: 7 - DNA, 18 bp, Artificial Sequence
  Synthetic Construct
actcgcgtgtacagccgg

SEQ ID NO: 8 - DNA, 19 bp, Artificial Sequence
  Synthetic Construct
agggatacccgtctccgtg

SEQ ID NO: 9 - DNA, 23 bp, Artificial Sequence
  Synthetic Construct
ccgcgacaccaaactggatgacg

SEQ ID NO: 10 - DNA, 20 bp, Artificial Sequence
  Synthetic Construct
tggcattgctgacatcatga

SEQ ID NO: 11 - DNA, 17 bp, Artificial Sequence
  Synthetic Construct
gccagagggcccatcaa

SEQ ID NO: 12 - DNA, 30 bp, Artificial Sequence
  Synthetic Construct
aagtcgccatgctccttaattccaaaagag

SEQ ID NO: 13 - DNA, 22 bp, Artificial Sequence
  Synthetic Construct
tgttgttgctgtggctgatagc

SEQ ID NO: 14 - DNA, 20 bp, Artificial Sequence
  Synthetic Construct
tctggtgtccccacgaactt

SEQ ID NO: 15 - DNA, 27 bp, Artificial Sequence
  Synthetic Construct
ttctgcaattccgacctcgtcatcagg

SEQ ID NO: 16 - DNA, 19 bp, Artificial Sequence
  Synthetic Construct
cagccgcttcacctacagc

SEQ ID NO: 17 - DNA, 20 bp, Artificial Sequence
  Synthetic Construct
tcaatcactgtcttgcccca

SEQ ID NO: 18 - DNA, 23 bp, Artificial Sequence
  Synthetic Construct
tcgatggctgcacgagtcacacc

SEQ ID NO: 19 - DNA, 23 bp, Artificial Sequence
  Synthetic Construct
tgactttgtcacaccccaagata

SEQ ID NO: 20 - DNA, 20 bp, Artificial Sequence
  Synthetic Construct
aatccaaatgcggcagcttc

SEQ ID NO: 21 - DNA, 27 bp, Artificial Sequence
  Synthetic Construct
tgatgctgcttacatgtctcgatccca

SEQ ID NO: 22 - DNA, 24 bp, Artificial Sequence
  Synthetic Construct
ccgtttccatctctctcaagatga

SEQ ID NO: 23 - DNA, 26 bp, Artificial Sequence
  Synthetic Construct
cagagagttagatttggtgggtacca

SEQ ID NO: 24 - DNA, 30 bp, Artificial Sequence
  Synthetic Construct
agatggtattcaatccctctatgaacctcc

SEQ ID NO: 25 - DNA, 25 bp, Artificial Sequence
  Synthetic Construct
ccgatgtatatgcttgcagagttaa

SEQ ID NO: 26 - DNA, 23 bp, Artificial Sequence
  Synthetic Construct
cagatgattcagagctccataga

SEQ ID NO: 27 - DNA, 27 bp, Artificial Sequence
  Synthetic Construct
aaccgtcacctgggaccgagacatgta